(19) United States
(12) Patent Application Publication
(10) Pub. No.: US 2013/0041426 A1
(43) Pub. Date: Feb. 14, 2013

(54) TRAINING/MEDICAL LOW-FREQUENCY ELECTROMYOSTIMULATING GARMENT

(71) Applicants: Anatoliy Ivanovich Grigoriev, Moscow (RU); Jury Andreevich Koriak, Orekhovo-Zuevo (RU); lnesa Benediktovna Kozlovskaya, Moscow (RU); Umberto Orazio Gluseppe Maugeri, Calvignano (IT); lrina Valerievna Sayenko, Naro-Fominsk (RU); Yevgenia Nikolaevna Yarmanova, Zhukovskiy (RU)

(72) Inventors: Anatoliy Ivanovich Grigoriev, Moscow (RU); Jury Andreevich Koriak, Orekhovo-Zuevo (RU); lnesa Benediktovna Kozlovskaya, Moscow (RU); Umberto Orazio Gluseppe Maugeri, Calvignano (IT); lrina Valerievna Sayenko, Naro-Fominsk (RU); Yevgenia Nikolaevna Yarmanova, Zhukovskiy (RU)

(21) Appl. No.: 13/261,337

(22) PCT Filed: Dec. 28, 2010
(86) PCT No.: PCT/EP2010/070788

§ 371 Date: Sep. 18, 2012

(30) Foreign Application Priority Data
Dec. 28, 2009 (RU) 2009148497

(51) Int. Cl. A61N 1/36; A61N 1/04

(52) U.S. Cl. 607/48; 607/149

(57) ABSTRACT

The training/medical electromyostimulating device according to the present invention is realized in the form of a garment to be worn by a person (PE); it comprises at least one electrical myostimulation signals generating device (EMS-LF-1, EMS-LF-2) adapted to generate electrical myostimulation signals at low pulse frequency; the or each device (EMS-LF-1, EMS-LF-2) is placed in the garment during normal use by the person (PE).

### FIELD OF THE INVENTION

The field of application of the present invention is training/medical devices to be used by astronauts during space flights of different duration or to be used for rehabilitation of patients suffering from muscle deficiency and/or decreased work capacity.

### PRIOR ART

Different constructions of garments for astronauts solving the human problems of lightening and adaptation to weightlessness are known from the prior art: for example, RU2254272, U.S. Pat. No. 4,051,848, RU2007146197.

But none of these constructions are completely satisfactory.

Compensative garments to be used in space flights are known e.g. from E. Vizleb “Functions of vascular system”, in the book “Human physiology”, 1996, V. 2, Ch. 20, p. 498-565.

But they do not provide sufficiently quick adaptation to and prolonged protection from decompensations on the astronauts body deriving from weightlessness.

A method of prophylaxis of weightlessness negative influence on human body is currently in use which includes application of negative pressure on the lower part of the body and dosed tractions of lower extremities by means of electrostimulated impulses.

The disadvantage of this method is the fact that stimulation is performed as a procedure with the only condition of keeping a tension below than the threshold of pain. Therefore, this solution suggests to fine-adjust the myostimulation application.

It has been found by the Applicant that it is advantageous to provide separate electromyostimulation of lower extremities and back muscles.

### SUMMARY OF THE INVENTION

The main object of the present invention is to develop a training/medical device designed to effectively electromyostimulate muscles of a human body.

This object is reached by the device having the technical features set out in the appended claims.

A first idea behind the present invention is to realize the device in the form of a garment to be worn by a person.

A second idea behind the present invention is to use low-frequencies, typically in the range from 1 Hz to 150 Hz.

A third idea behind the present invention is to provide separate stimulations of muscles, in particular of back and lower extremities muscles and/or right and left muscles and/or a single muscle or group of muscles.

A fourth idea behind the present invention is to use two electromyostimulators, i.e. electrical myostimulation signals generating devices.

A fifth idea behind the present invention is to provide a garment with integral system of fixation of the or each generating devices to the body of the person.

A sixth idea behind the present invention is to provide a garment with integral electrical contacts for applying the electrical myostimulation signals to the body of the person.

Advantageously, all these ideas are implemented in a device according to the present invention; other ideas and technical features are not to be excluded.

The person who wears the garment according to the present invention will be subject to electromyostimulation treatments; each treatment consists in the cyclic application to the body of the person of a electromyostimulation signals for a certain amount of time; the signals may vary during the treatment (for example, at first signals will be applied to the lower right limb muscles and then to the lower left limb muscles); one or more treatments may occur during e.g. a single hour or day or week or month; in general the treatments for a person will decided by a doctor.

### BRIEF DESCRIPTION OF THE DRAWINGS

The present invention can be better understood together with its technical features and advantages from the following detailed description to be considered together with the appended drawings, wherein:

FIG. 1 is a block diagram of an embodiment of the present invention,

FIG. 2 is a block diagram showing the possibility of connecting an electromyostimulator of the embodiment of FIG. 1 with a personal computer, and

FIG. 3 shows, in a front view (left image) and a back view (right image), schematically possible electrical connections to a body.

The following description is not to be considered restrictively especially because it refers only to a specific (even if advantageous) embodiment of the present invention.

### DETAILED DESCRIPTION OF THE INVENTION

The training/medical low-frequency electromyostimulating device, in the form of a garment, according to the present invention, comprises at leas one but preferably two electrical myostimulation signals generating devices—in the following, each of them will be referred to also as “electromyostimulator” or “EMS-LF”; electromyostimulators can work in parallel, i.e. simultaneously, and they are placed into holding elements of a fixation system of the garment so that they can be located on the right and on the left of a human body (typically and advantageously, one on the right and one on the left); electromyostimulators are directly connected to cables of the garment that lead to electrodes of the garment so that electromyostimulation signals can start from the electromyostimulators go through the cables and end to the electrodes placed on and in contact with some muscles or groups of muscles; generated electromyostimulation signals are synchronized; typically, the two electromyostimulators are connected for such synchronization after switch-on; typically, the main electromyostimulator is placed on the right of the body; there is the possibility to adjust at least the amplitude of the electromyostimulation signal; additionally and advantageously, there is the possibility of transferring and storing from one or any or both of the electromyostimulators into a PC [Personal Computer] various information through e.g. a serial communication interface, such as a RS-232 interface; the information to be transferred may by selected by the user, for example the astronaut or the patient or, more typically, the doctor, a may correspond e.g. to values of parameters (for example the amplitude) of the electromyostimulation signals of a treatment of a person, and/or to the date and time of treatment of a person, and/or to the personal details of a treated person.

The garment according to the present invention is advantageously provided with autonomous power source from supply batteries that may be placed inside the electromyostimulators; in this way, the treatment may be carried out in any place and at any time. Additionally and further advantageously, an intelligent charger is provided for charging these batteries.

As it is provided that the maximum treatment/activity-period duration is 6 hours, the garment is a structurally designed accordingly; for example, the electromyostimulators switch-off automatically after 6 hours of operation, the batteries of the electromyostimulators are chosen so to last for 6 hours (or more), the storage capacity of data inside the electromyostimulators is chosen so to be able to store information corresponding to 6 hours (or more) of treatment/activity-period (thereafter, all information may be transferred to a PC so that the freed storage capacity will be available for further storage).

The garment according to the present invention allows to perform separate stimulation of back muscles and lower extremities muscles, of right muscles and left muscles; advantageously, it has been provided for separate stimulation of single muscles or groups of muscles; in this way, the treatment may be customized to the needs of any single person. The garment according to the present invention is advantageously provided with a moistening device for moistening the electromyostimulation electrodes; in this way, the electric contact between the electrodes and the body is always very good even if a long time passes between two consecutive treatments and/or if the person under treatment is not within a treatment environment (such as a clinic) but may be in any place of a house or of an office.

A very important aspect of the present invention is that the electromyostimulation system takes the form of a garment so that it can be carried by the patient; the treatments may be repeated even outside of a clinic and at any time of the day or night according to the best treatment strategy avoiding any other limitation; to this regard, the fixation system to the body, electrodes in the form of tissue, the long-duration batteries, the moistening device for electrodes play a very important role.

Another important aspect of the present invention is the starting of the treatments; this can be automatic, i.e. triggered by the stimulator by means of its internal circuitry and/or of external detectors, or triggered by the user or a combination of the two things. As it is clear, the treatment, i.e. the generation of the signals during the treatment period, is under the full control of the stimulator.

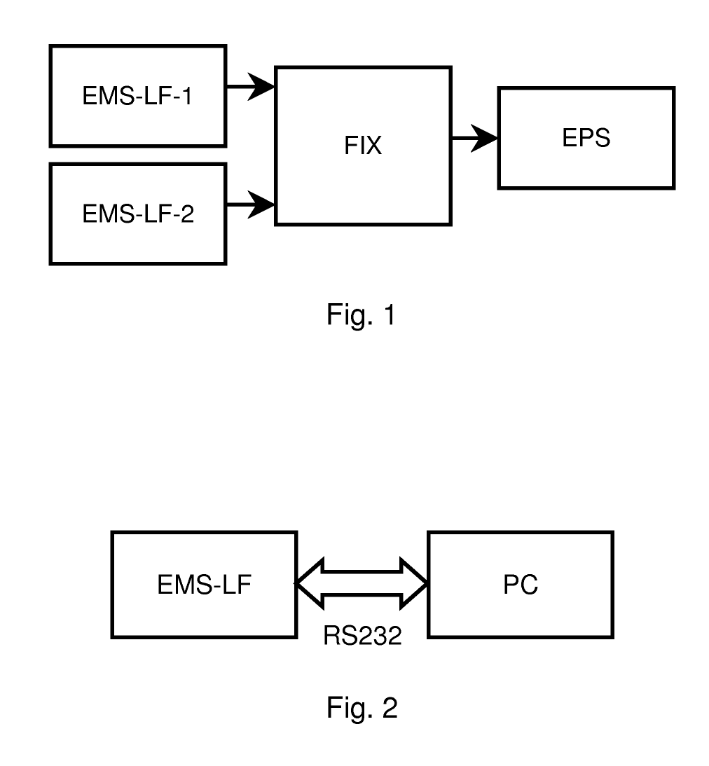

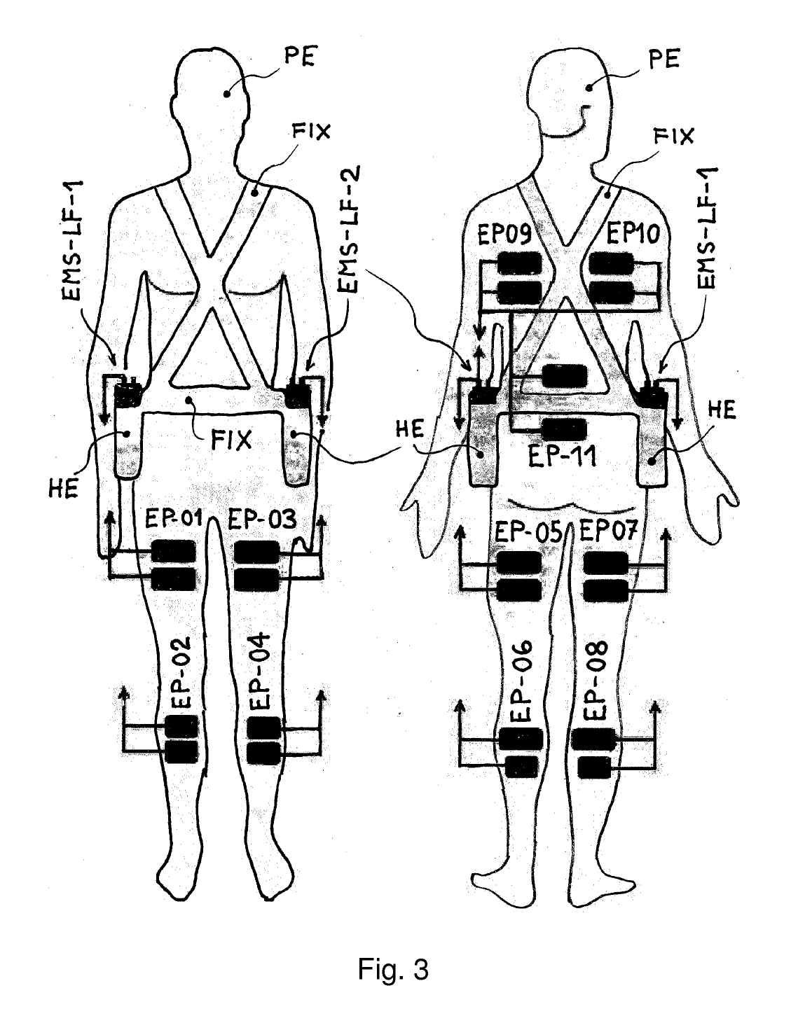

In the following a detailed description of a specific example of a garment and its components, including low-frequency electromyostimulators or EMS-LFs, is provided; reference will be specifically made to FIG. 1, FIG. 2, FIG. 3.

The block diagram of FIG. 1 shows two electrical myostimulation signals generating devices EMS-LF-1 and EMS-LF-2 of the garment; any of them is labeled EMS-LF as in FIG. 2. The devices EMS-LF-1 and EMS-LF-2 are placed into holding elements HE of a fixation system FIX of the garment so that they can be located on the right and on the left of the body of a person PE (see FIG. 3). The devices EMS-LF-1 and EMS-LF-2 are directly connected to cables of the garment that lead to electrodes EPS of the garment; electrodes are provided in pairs for the flow of electric current between them across the body, specifically the muscles, of the person. In FIG. 3, eleven pairs of electrodes labeled from EP-01 to EP-11 are provided: electrodes EP-01 serve to stimulate front muscles of upper part of left leg, electrodes EP-02 serve to stimulate front muscles of lower part of left leg, electrodes EP-03 serve to stimulate front muscles of upper part of right leg, electrodes EP-04 serve to stimulate front muscles of lower part of right leg, electrodes EP-05 serve to stimulate rear muscles of upper part of right leg (right thigh), electrodes EP-06 serve to stimulate rear muscles of lower part of right leg (right calf), electrodes EP-07 serve to stimulate rear muscles of upper part of left leg (left thigh), electrodes EP-08 serve to stimulate rear muscles of lower part of left leg (left calf), electrodes EP-09 serve to stimulate the muscles of the right shoulder blade, electrodes EP-10 serve to stimulate the muscles of the left shoulder blade, electrodes EP-11 serve to stimulate the muscles of loin. In the example of these figures, device EMS-LF-1 drives electrodes on one lower limb EP-01, EP-02, EP-07, EP-08 and device EMS-LF-2 drives electrodes on the other lower limb EP-03, EP-04, EP-05, EP-06, as well as those on the back EP-09, EP-10, EP-11.

FIG. 2 shows the possibility of connecting any of the devices EMS-LF-1 and EMS-LF-2 of FIG. 1 to a personal computer PC for transferring data from the device, labeled generally as EMS-LF, to the personal computer PC; before transfer these data are stored in a FLASH memory of device EMS-LF; after transfer these data are stored in a Hard-Disk of computer PC. In this example, the transferred is carried out through serial communication means, i.e. a RS-232 cable and appropriate interfaces in device EMS-LF and in computer PC.

The time for starting the EMS-LF is not more than one minute.

Duration of EMS-LF maximal continuous operation (i.e. proving stimulation signals) is not less 6 hours.

EMS-LF meets the requirements of the (Russian) State Standard P 50267.0-92 (IEC 601-1), and of (Russian) State Standard P 50267.10-93 (IEC 601-2-10) for electro-safety and belongs to:
products/parts with internal power source of II class as far as protection from electrical shock regards;
products/parts of BF type as far as protection level regards.

Maximum values of the overall dimensions and mass of the EMS-LF elements are:
overall dimensions in the case (without garment): not more than 410×300×70 mm;
overall dimensions of the garment in the case: not more than 400×320×95 mm;
mass of the EMS-LF in the case (without garment): 3.8±0.1 kg;
mass of the garment with electrodes in the case: 2.3±0.1 kg.

Electromyostimulation signals come through the cables placed in the garment to the electrodes placed on and in contact with the muscles or groups of muscles.

There is also a cable in the garment which connects the two EMS-LFs with each other and provides synchronization of combined operation of both EMS-LFs after switching-on.

EMS-LFs are placed into holding elements of the fixation system of the garment and they are directly connected to cables of the garment (that lead to electrodes of the garment) in strict accordance with markings on the garment/EMS-LF connectors. Each EMS-LF has six channels of electromyostimulation with the following characteristics:
operation mode: cyclic;
maxim duration of stimulation: 6 hours;
stimulation: 1±0.1 sec (duration of pulses bursts);
pause: 2±0.1 sec (duration of the time interval between pulses bursts); —type of signal: bipolar symmetric rectangular pulse;
duration of pulses: 1±0.05 ms;
frequency of pulses: 25±1 hertz;
frequency of current during a burst: 1-10 KHz;
amplitude of the electromyostimulation signal at load resistance of 500 ohm: not more than 45 V;
error of the setting of amplitude of electromyostimulation signal: not more than ±10% (without taking into account the error of the equipment controlling the signal).

Stimulators of other garments (alternative to this one) might have different characteristics or more widely adjustable characteristics; for example, possible and advantageous values of the stimulation are from 0.5 sec to 2 sec, possible and advantageous values of the pause are from 0.5 sec to 5 sec, possible and advantageous values of duration of pulse are from 0.2 ms to 2.0 ms, possible and advantageous values of frequency of pulses are from 10 Hz to 50 Hz.

The possibility of adjusting the amplitude of electromyostimulation signals separately for each channel is provided in the EMS-LF; during adjusting one channel, other channels of electromyostimulation are switched-off.

The EMS-LF provides the storage of information for one 6-hours treatment/activity-period. The EMS-LF has onboard graphical liquid-crystal display (LCD) with a resolution of 128×64 pixels; information displayed on the LCD contains:
information about the on-going treatment of the EMS-LF: amplitude of the electromyostimulation signal (with graphic and digital indication of its value) and stimulation duration (hr, min, sec);
information about start time and end time of the treatment.

All captions on the display are made in two languages, namely Russian (manufacturer language) and English (user language).

Power supply of the EMS-LF is from an autonomous power source: storage battery (SB) NiMH, with nominal voltage of 10.8 V and capacity of 1.7 Ah.

Construction of the EMS-LF excludes the possibility of getting inside any aperture and also of accidental touch to internal elements.

Overall sizes of the EMS-LF are not more than 212×111×45 mm.

Mass of the EMS-LF with SB is 1.25±0.1 kg.

EMS-LF consists of printed circuit boards (i.e. sub-units or sub-systems):
switching device;
controller;
power amplifier;
commutator.

The switching device is intended for the forming of input signals to the controller carrying information about the EMS-LF operation, selection of the electromyostimulation channel number, increasing or decreasing of the amplitude of the electromyostimulation signal. The controller is intended for the control of the operation of all EMS-LFs and provides:
forming of electromyostimulation signals;
adjusting of the amplitude of the electromyostimulation signals in the power amplifier;
forming of the information to be displayed on the LCD;
recording and storage into a FLASH memory of the following information: date and time of start and end of electromyostimulation, value of the initially selected amplitude of the electromyostimulation signal for every channel, time of change of the amplitude of the electromyostimulation signal at any channel and its new value;
transfer of information stored in the FLASH memory to a PC by a standard RS-232 interface;
control of commutator operation.

The power amplifier is controlled by the controller and is intended for the forming of electromyostimulation signals, adjusting of their amplitude and providing of galvanic decoupling of output electromyostimulation channel circuits.

The power amplifier consists of two channels for signal amplification with two isolation transformers.

The EMS-LF includes a charger (CH) intended for simultaneous charge of two SBs. Power voltage at the CH comes from an external power source with a voltage of 28 V.

CH is “intelligent” and does not require human control during charging: charging stops automatically so protection from overcharge of the SBs is guaranteed. During operation of the CH, a LED indication of all its functions is provided.

The training-medical low-frequency electromyostimulation garment could be additionally provided a moistening device for moistening of the electromyostimulation electrodes.

The EMS-LF contains different treatment programs than can be selected by the user (for example the astronaut or the patient or the doctor); the following programs are provided in this example:
program 1: stimulation of lower right limbs,
program 2: stimulation of lower left limbs,
program 3: contemporaneous stimulation of lower right and left limbs,
program 4: stimulation of back.

For program 3, two EMS-LF are used.

As it is clear more complex programs may be provided or programmed by a doctor or a technician.

### Data Transfer Mode

The following information is recorded in the FLASH memory of the EMS-LF during electromyostimulation:
date of electromyostimulation;
value of the initially selected amplitude of the electromyostimulation signal for every channel;
time of start and end of the electromyostimulation;
time of change of the amplitude of the electromyostimulation signal at any channel and its new value.

The EMS-F provides the storage of this information (one 6-hours treatment/activity-period).

To transfer information into a PC, the EMS-LF should be switched on and a software (STIMUL-01) should be installed in the PC according to its installing instructions contained in an appropriate guide.

The STIMUL-01 software is started according to the instructions of the guide, both the EMS-LFs are to be connected to the PC in turn according to the instructions of the guide. At this time, information stored in the FLASH memory of the EMS-LF is transferred to the PC and stored into its hard disk for further use; thereafter, the FLASH memory of the EMS-LF is cleaned.

The EMS-LF is intended for the forming of electromyostimulation signals and also for recording of information, storage into FLASH memory and transfer to PC of the information about performed electromyostimulation treatments.

The use of EMS-LF for its intended purpose:

EMS-LF has the following modes of use:
electromyostimulation mode;
charging of SB mode;
mode of information read-out from EMS-LF to PC through the interface RS-232;
training mode (charge-discharge) of SB.

1. Training/medical electromyostimulating device realized in the form of a garment to be worn by a person comprising at least one electrical myostimulation signals generating device adapted to generate electrical myostimulation signals at low pulse frequency, wherein the or each device is placed in the garment during normal use by the person.

2. Garment according to claim 1, comprising two electrical myostimulation signals generating devices being both placed in the garment during normal use by the person.

3. Garment according to claim 2, wherein the two devices are adapted to work contemporaneously and are electrically connected so to be synchronized with each other.

4. Garment according to claim 1, comprising further an integral system of fixation of the or each device to the body of the person, in particular on the right and/or on the left of the body of the person.

5. Garment according to claim 1, comprising further integral cables for transferring electrical myostimulation signals from the or each device to electrodes adapted to apply them to the body of the person.

6. Garment according to claim 1, comprising further integral electrodes for applying the generated electrical myostimulation signals to the body of the person.

7. Garment according to claim 5, comprising further a moistening device for moistening said electrodes.

8. Garment according to claim 1, wherein the or each device comprises supply batteries.

9. Garment according to claim 8, comprising further a battery charger, in particular an intelligent battery charger, for charging the batteries of the or each device.

10. Garment according to claim 1, wherein the or each device comprises further means for storing information relating to the carried-out treatments and means for transferring the stored information to a computer.

11. Garment according to claim 10, wherein the information to be stored and/or transferred is selected by a user.

12. Garment according to claim 1, wherein the or each device controls the following parameters of the cyclic generated electrical myostimulation signals:
duration of stimulation;
duration of pause;
duration of pulses;
frequency of pulses;
frequency of current during bursts;
amplitude of signals.

13. Garment according to claim 12, wherein one or more of said parameters are adjustable by user, in particular the amplitude of signals.

14. Garment according to claim 1, wherein the or each device has a plurality of independent channels for electrical myostimulation signals.

15. Garment according to claim 14, wherein each of the channels may be adjusted separately.

16. Garment according to claim 1, wherein the or each device is adapted for a maximal duration of low-frequency electromyostimulation of six hours.

17. Garment according to claim 1, being adapted for separate stimulations, in particular of back and lower extremities muscles and/or right and left muscles and/or a single muscle or group of muscles.

18. Garment according to claim 1, wherein the or each device has pre-stored treatment programs.

19. Garment according to claim 1, wherein the starting of a treatment is triggered by the or each device by means of its internal circuitry and/or of external detectors.

20. Garment according to claim 1, wherein the starting of a treatment is triggered by a user.
